(19) United States
(12) Patent Application Publication
(10) Pub. No.: US 2025/0387190 A1
(43) Pub. Date: Dec. 25, 2025

(54) ATTACHABLE MEDICAL DEVICE

(71) Applicant: Christopher Zarembinski, Los Angeles, CA (US)

(72) Inventor: Christopher Zarembinski, Los Angeles, CA (US)

(21) Appl. No.: 19/247,456

(22) Filed: Jun. 24, 2025

Related U.S. Application Data
(60) Provisional application No. 63/663,805, filed on Jun. 25, 2024.

(51) Int. Cl. A61B 50/20

(52) U.S. Cl. CPC A61B 50/20

(57) ABSTRACT

Provided herein are devices attachable to medical devices. In some embodiments, these devices are used to guide positioning of needles and/or limit migration of needles. In some embodiments, these devices are used to improve pull out strength by being snapped into place. The device includes a first body, a second body, and a bridge connecting the first and second bodies. A gap is formed on top of the bridge and in-between the first body and the second body. The device further includes a first slit formed at the first body and a second slit formed at the second body. A first canal including portions of the gap, first slit, and second slit, and passing centrally through the device is formed. A second canal is formed on top of the bridge and at a bottom portion of the gap, the second canal passing centrally through the device.

### CROSS-REFERENCE TO RELATED APPLICATIONS

This application claims priority to and the benefit of U.S. Provisional Application No. 63/663,805, filed on Jun. 25, 2024, which is incorporated herein by reference in its entirety.

### FIELD OF INVENTION

This invention relates generally to a medical device attachable to various types of medical devices, and, more specifically, to a medical device attachable to various type of needles or various types of leads for a pacemaker, an implantable cardioverter-defibrillator (ICD), a spinal cord stimulator lead, or an intrathecal drug delivery catheter.

### BACKGROUND OF THE INVENTION

### Needle Stop

Radiofrequency ablation (RFA) is a minimally invasive treatment for localized cancer. RFA is used to treat benign and cancerous tumors. RFA destroys tumors in a less invasive way compared to traditional surgical removal.

In operation, a needle with a plastic-insulated shaft is placed into the tumor with imaging guidance, in a fashion similar to a routine percutaneous biopsy. A slender needle-like probe is inserted into the body and placed directly into the tumor.

For percutaneous RFA, a needle-sized electrode is passed directly through the skin and guided into place. Thus, the procedure of RFA includes directing a needle into a desired location. During the procedure, the needle is observed and positioned by a medical professional in real-time to ensure that the needle reaches the desired location. However, the current procedure is problematic because visual observation of the needle is not sufficient to prevent erroneous needle positioning. Once the needle is in place, a radiofrequency current is passed through the needle. Upon completion of the treatment, the tumor is destroyed and will no longer enhance on CT imaging. Patients would benefit from percutaneous RF ablation, particularly in the brain, liver, lung, pancreas, kidney, and uterus. The primary indication for RF ablation is tumor ablation.

In order to achieve effective results, accuracy of positioning an RFA electrode in a target site is important. However, accurate needle placement in tumors can be difficult. For example, during needle progression toward a particular target, body position changes can affect accurate targeting. A needle guidance technology for accurate needle positioning in the target site would be beneficial for successful RFA.

Accurate positioning of a needle in a target site is also important to limit migration of the needle intraoperatively and to enhance safety. In another application of the subject technology, endo file drills used in root canal surgery also need to be positioned in a target site accurately. For example, FIG. 8 shows a prior art device attached to endo file drills used in root canal surgery to be used as a stopper. If a needle or endo file drill migrates, then injury could result. The present disclosure provides a solution for these and other problems.

A deployable needle stop, as described in the present disclosure, may limit inadvertent needle migration and improve the safety of a surgical or other medical intervention. For example, the deployable needle stop may grab the needle by snapping into place as a single unit and preventing unwanted needle migration. The needle stop may grab the shaft of the needle adjacent to the skin entry point and prevent migration, thereby improving safety in procedures involving needles.

### Anchor Sleeve

Securing a lead or catheter to a device pocket at implantation may prevent lead dislodgement. A sleeve anchors the lead to the pocket floor and may prevent it from being pulled out or substantially reduces the possibility thereof. The sleeve does this by gripping the lead body and holding it down to the pocket floor via sutures. The role of the sleeve is to hold the leads until the enveloping layer of tissue forms around the lead.

An anchor should be able to securely hold a catheter without substantially crushing, kinking, pinching or occluding the catheter. Further, an anchor may be securely attached to a patient's tissue. An anchor sleeve that snaps into place may minimize lead injury and improves pull out strength.

A novel anchor sleeve, as described in the present disclosure, grabs the lead by snapping into place. The novel anchor sleeve avoids circumferential deformation of the sleeve caused by encircling suture, thereby minimizing damage to the lead. Further, by increasing contact with the lead, pull out strength may be enhanced. For example, the novel anchor sleeve may secure lead placement for a pacemaker, an implantable cardioverter-defibrillator (ICD), a spinal cord stimulator lead, or an intrathecal drug delivery catheter. A snap on anchor sleeve may minimize revision of an implanted lead that may be caused by damage to the lead or migration of the lead. The novel anchor sleeve disclosed herein could become a standard in pacemaker or ICD implementation.

### SUMMARY OF THE INVENTION

According to certain aspects of the present disclosure, a device configured to receive a medical device is disclosed. According to various embodiments, the device includes a bridge; a first body connected to a first side of the bridge, the first body having a generally hemisphere shape and including a first surface and a second surface, the first surface being a non-flat curved surface, the second surface being in the form of a flat circle; and a second body connected to a second side of the bridge, the second body having a generally hemisphere shape and including a third surface and a fourth surface, the third surface being a non-flat curved surface, the fourth surface being in the form of a flat circle. A gap is formed on top of the bridge and in-between the second surface of the first body and the fourth surface of the second body. The gap is configured for receiving within at least a portion of a medical device. When the medical device is at least partially inserted into a target site of a subject, at least one of the first body and the second body prevents contact between (i) at least a portion of the medical device and (ii) the target site.

In various embodiments, the overall shape of the device is generally spherical, and wherein the device is visible as a circular shape according to a top view or a bottom view of the device.

In various embodiments, the first body, the gap, and the second body are visible according to a top view of the device, and wherein the gap is not visible according to a bottom view of the device, the bottom view being opposite the top view of the device.

In various embodiments, the first body has a first slit and the second body has a second slit.

In various embodiments, the first slit and the second slit are not visible according to the top view or the bottom view of the device.

In various embodiments, the gap and the first slit are visible and the second slit is not visible according to a front view of the device, the first body and the second body being visible according to the front view. In some embodiments, the gap and the second slit are visible and the first slit is not visible according to a rear view of the device that is opposite the front view of the device, the first body and the second body being visible according to the rear view.

In various embodiments, the entire first slit is visible and the second slit is not visible entirely according to a right side view of the device, the first body being visible and the second body not being visible according to the right side view. In some embodiments, the device is visible as a circle or as a shape similar to a circle according to the right side view of the device. In some embodiments, the entire second slit is visible and the first slit is not visible entirely according to a left side view of the device that is opposite the right side view of the device, the second body being visible and the first body not being visible according to the left side view. In some embodiments, the device is visible as a circle or as a shape similar to a circle according to the left side view of the device.

In various embodiments, the first slit has an opening formed toward a first direction and the second slit has an opening formed toward a second direction that is opposite the first direction. In some embodiments, a first open space is formed by a first portion of the gap and the first slit and a second open space is formed by a second portion of the gap and the second slit.

In various embodiments, the first open space and the second open space overlap partially such that a first canal is formed, the first canal including the overlapping space and passing centrally through the device. In some embodiments, a top side of the bridge is curved inwardly such that a second canal is formed on top of the bridge and at a bottom portion of the gap, the second canal being a portion of the gap and passing centrally through the device. In some embodiments, the second canal also includes the overlapping space and the first canal is perpendicular to the second canal. In some embodiments, the first canal and the second canal intersect each other at a point corresponding to a center of the device, the device generally having a shape of a sphere or an overall shape of the device being similar to a sphere. In some embodiments, the first slit and the second slit are positioned at opposite sides relative to the first canal.

In various embodiments, the first slit and the second slit are positioned at opposite sides relative to the second canal.

In various embodiments, the first canal is visible as an opening, a through hole, or an empty space according to the right side view of the device, the second body not being visible according to the right side view of the device. In some embodiments, the first canal is visible as an opening, a through hole, or an empty space according to the left side view of the device, the first body not being visible according to the left side view of the device.

In various embodiments, the medical device includes a needle or an endo file. The device is configured to receive the needle or endo file such that the received needle or endo file passes through the first canal. The first canal has a ribbed surface configured to grip the received need or endo file.

In various embodiments, a diameter of the first canal is at least about 0.5 millimeter (mm) and less than about 2.5 mm, about 0.5 mm, about 0.6 mm, about 0.7 mm, about 0.8 mm, about 0.9 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.1 mm, or about 2.5 mm.

In various embodiments, a diameter of the second canal is at least about 1 millimeter (mm) and less than about 3 mm or 4 mm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.1 mm, about 2.5 mm, about 2.6 mm, about 2.7 mm, about 2.8 mm, about 2.9 mm, or about 3.0 mm.

In various embodiments, the device is further configured to stop the needle or endo file being inserted into the target site by blocking further insertion.

In various embodiments, the needle comprises a radiofrequency (RF) needle. In various embodiments, the endo file is a drill used in root canal surgery.

In various embodiments, a diameter of the first body and a diameter of the second body are same. In various embodiments, a width of the device and a length of the device are same.

In various embodiments, a distance between a first side body and a second side body corresponding to a width of the gap is at least about 1 millimeter (mm) and less than about 3 mm or 4 mm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.1 mm, about 2.5 mm, about 2.6 mm, about 2.7 mm, about 2.8 mm, about 2.9 mm, or about 3.0 mm.

In various embodiments, a width of the device corresponding to a length of the gap is at least about 10 mm and less than about 13 mm, about 10 mm, about 10.2 mm, about 10.4 mm, about 10.6 mm, about 10.8 mm, about 11.0 mm, about 11.2 mm, about 11.4 mm, about 11.6 mm, about 11.8 mm, about 12.0 mm, about 12.2 mm, about 12.4 mm, about 12.6 mm, about 12.8 mm, or about 13.0 mm. In various embodiments, a length of the device is at least about 10 mm and less than about 13 mm, about 10 mm, about 10.2 mm, about 10.4 mm, about 10.6 mm, about 10.8 mm, about 11.0 mm, about 11.2 mm, about 11.4 mm, about 11.6 mm, about 11.8 mm, about 12.0 mm, about 12.2 mm, about 12.4 mm, about 12.6 mm, about 12.8 mm, or about 13.0 mm.

In various embodiments, the device is made of biocompatible materials, medical grade materials suitable for medical devices, or materials that are currently accepted by the U.S. Food and Drug Administration (FDA) for use in medical devices, the materials including, but not limited to silicones, polymers, metals, ceramics, rubber, plastics, or any combination thereof.

### BRIEF DESCRIPTION OF DRAWINGS

Exemplary embodiments are illustrated in the referenced figures. It is intended that the embodiments and figures disclosed herein are to be considered illustrative rather than restrictive.

FIG. 1A depicts, in accordance with various embodiments of the present invention, a top view of an attachable needle stop device.

FIG. 1B depicts, in accordance with various embodiments of the present invention, a front perspective view of an attachable needle stop device.

FIG. 1C depicts, in accordance with various embodiments of the present invention, a front view of an attachable needle stop device.

FIG. 1D depicts, in accordance with various embodiments of the present invention, a right side view of an attachable needle stop device.

FIG. 2 depicts, in accordance with various embodiments of the present invention, exemplary dimensions of an attachable needle stop device.

FIG. 3 depicts, in accordance with various embodiments of the present invention, various views of an attachable needle stop device.

FIG. 4 depicts, in accordance with various embodiments of the present invention, various views of an attachable needle stop device.

FIG. 5 depicts, in accordance with various embodiments of the present invention, various views of an attachable needle stop device.

FIG. 6 depicts, in accordance with various embodiments of the present invention, various views of an attachable needle stop device.

FIG. 7 depicts, in accordance with various embodiments of the present invention, various views of an attachable needle stop device.

FIG. 8 shows a prior art device attached to endo file drills used in root canal surgery to be used as a stopper.

FIG. 9A depicts, in accordance with various embodiments of the present invention, a top view of an attachable anchor sleeve device.

FIG. 9B depicts, in accordance with various embodiments of the present invention, a front perspective view of an attachable anchor sleeve device.

FIG. 9C depicts, in accordance with various embodiments of the present invention, a front view of an attachable anchor sleeve device.

FIG. 9D depicts, in accordance with various embodiments of the present invention, a right side view of an attachable anchor sleeve device.

FIG. 10 depicts, in accordance with various embodiments of the present invention, an exemplary dimension of an attachable anchor sleeve device.

FIG. 11 depicts, in accordance with various embodiments of the present invention, various views of an attachable anchor sleeve device.

FIG. 12 depicts, in accordance with various embodiments of the present invention, various views of an attachable anchor sleeve device.

FIG. 13 depicts, in accordance with various embodiments of the present invention, various views of an attachable anchor sleeve device.

FIG. 14 depicts, in accordance with various embodiments of the present invention, various views of an attachable anchor sleeve device.

FIG. 15 depicts, in accordance with various embodiments of the present invention, various views of an attachable anchor sleeve device.

FIG. 16 depicts, in accordance with various embodiments of the present invention, various views of an attachable anchor sleeve device.

### DETAILED DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs.

One skilled in the art will recognize many methods and materials similar or equivalent to those described herein, which could be used in the practice of the present invention. Indeed, the present invention is in no way limited to the methods and materials described.

### Needle Guide/Stop

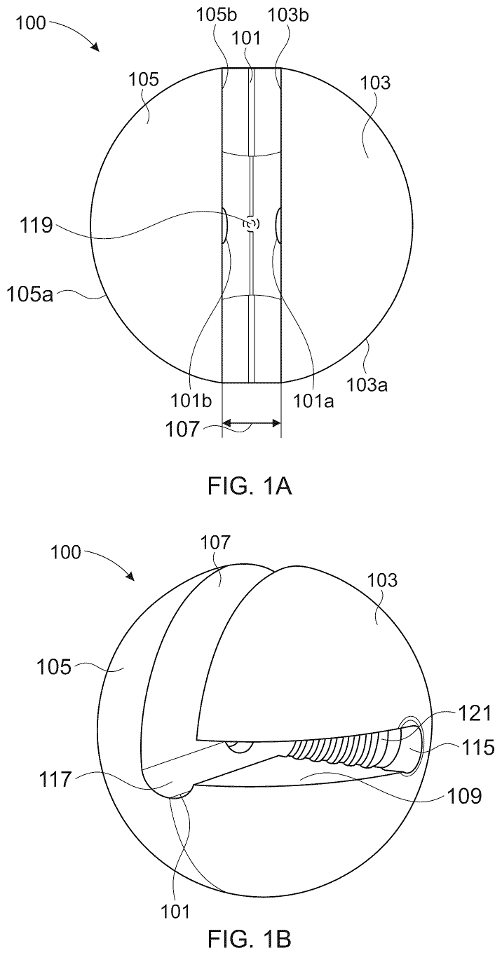

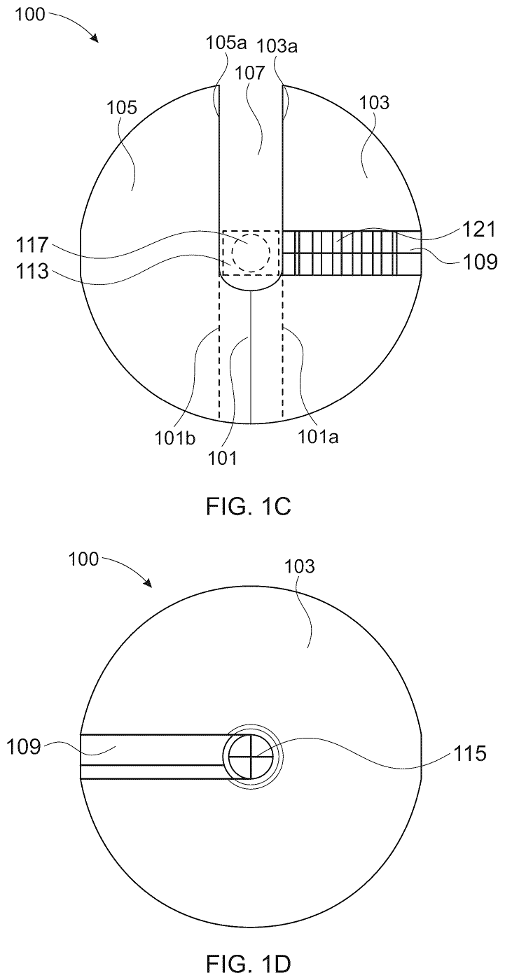

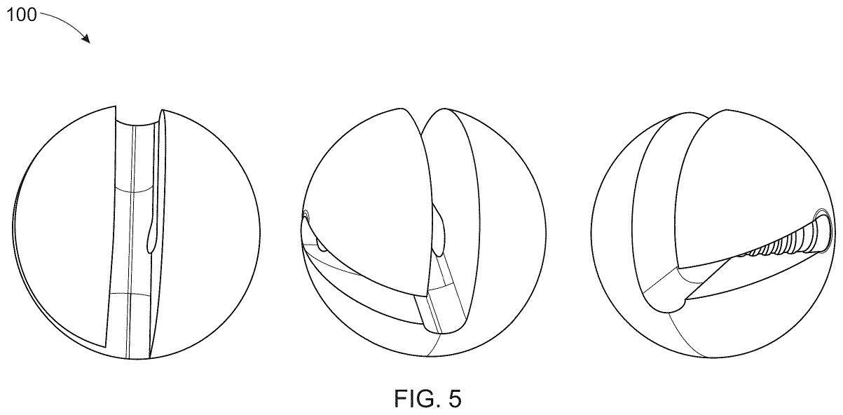

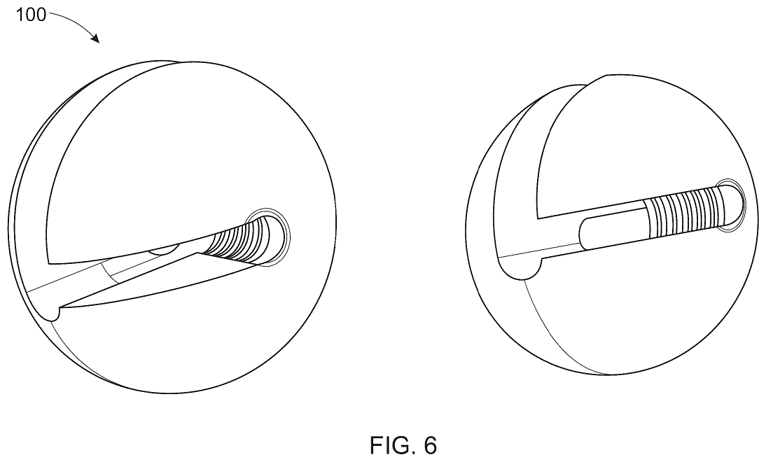

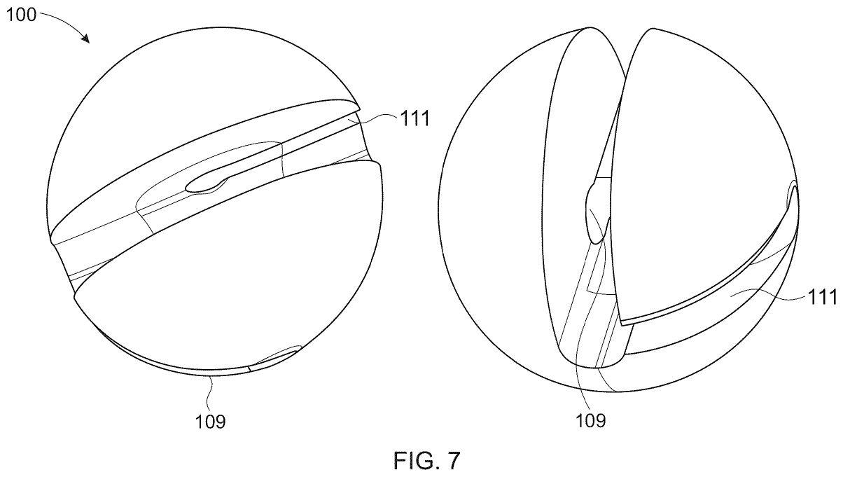

Provided herein are various embodiments of a device 100 for guiding/stopping needles to limit needle migration. According to one embodiment, the device 100 is configured to be attached or coupled to a needle. Referring to FIGS. 1A-7, the device 100 includes a bridge 101, a first body 103, and a second body 105. The first body 103 is connected to a first side 101a of the bridge 101 and has a generally hemispherical shape or a shape similar to a hemisphere, although other shapes and configurations may be used in alternate embodiments of the invention. The first body 103 includes a first surface 103a that is generally a non-flat, curved surface, and a second surface 103b that is generally flat and that may optionally have a generally circular shape. For example, the second surface 103b is in the form of a flat circle.

The second body 105 is connected to a second side 101b of the bridge 101 and has a generally hemisphere shape or a shape similar to a hemisphere, although other shapes and configurations may be used in alternate embodiments of the invention. The second body 105 includes a third surface 105a that is generally a non-flat, curved surface, and a fourth surface 105b that is generally flat and has a generally circular shape. For example, the fourth surface is in the form of a flat circle. Each of the first body 103 and the second body 105 has a slit formed therein, a first slit 109 and a second slit 111, respectively. The first slit 109 is shown in FIGS. 1B-1D, and the second slit 111 is shown in FIG. 7.

A gap 107 is formed on top of the bridge 101 and in-between the second surface 103b of the first body 103 and the fourth surface 105b of the second body 105. The overall shape of the device 100 is generally spherical, or a general shape of the device 100 is similar to a sphere.

In various embodiments, the device 100 is visible as a circle or as a generally circular shape according to a top view or a bottom view of the device 100, although other shapes and configurations may be used in alternate embodiments of the invention. For example, see FIG. 1A. The first body 103, the gap 107, and the second body 105 are visible according to the top view of the device 100, as shown in FIG. 1A. The gap 107 is not visible according to the bottom view of the device 100 (not shown in drawings). The bottom view is opposite the top view of the device 100 shown in FIG. 1A. The first slit 109 and the second slit 111 are not visible according to the top view or the bottom view of the device 100. See FIG. 1A, for example. The gap 107 and the first slit 109 are visible and the second slit 111 are not visible according to a front view of the device 100. See FIG. 1C. The first body 103 and the second body 105 are visible according to the front view, as shown in FIG. IC. The gap 107 and the second slit 111 are visible and the first slit 109 are not visible according to a rear view of the device 100 that is opposite the front view of the device 100 shown in FIG. 1C. The first body 103 and the second body 105 are visible according to the rear view. The entire first slit 109 is visible and the second slit 111 is not visible entirely according to a right side view of the device 100, as shown in FIG. 1D. The first body 103 is visible and the second body 105 is not visible according to the right side view. The device 100 is visible as a circle or as a generally circular shape according to the right side view of the device 100, although other shapes and configurations may be used in alternate embodiments of the invention. The entire second slit 111 is visible and the first slit 109 is not visible entirely according to a left side view of the device 100 that is opposite the right side view of the device 100 shown in FIG. 1D. The second body 105 is visible and the first body 103 is not visible according to the left side view. The device 100 is visible as a circle or as a generally circular shape according to the left side view of the device 100, although other shapes and configurations may be used in alternate embodiments of the invention.

The first slit 109 has an opening formed toward a first direction (toward the front of the device 100) and the second slit 111 has an opening formed toward a second direction (toward the rear of the device 100) that is opposite the first direction. A first open space is formed by a first portion 113 of the gap 107 and the first slit 109 and a second open space (not shown clearly in drawings) is formed by a second portion of the gap 107 and the second slit 111. The first open space and the second open space overlap partially such that a first canal 115 is formed. The first canal 115 includes the overlapping space and pass centrally through the device 100, as shown in FIG. 1B.

A top side of the bridge 101 is curved inwardly, as exemplified in FIG. 1B, such that a second canal 117 is formed on top of the bridge 101 and at a bottom portion of the gap 107. The second canal 117 is a portion of the gap 107 and passes centrally through the device 100. The second canal 117 also includes the overlapping space and the first canal 115 is generally perpendicular to the second canal 117. The first canal 115 and the second canal 117 intersect each other at a point generally corresponding to a center 119 of the device 100. The device 100 generally has a spherical shape or an overall shape of the device 100 is generally a spherical shape, although other shapes and configurations may be used in alternate embodiments of the invention. The first slit 109 and the second slit 111 are generally positioned at opposite sides relative to the first canal 115. The first slit 109 and the second slit 111 are generally positioned at opposite sides relative to the second canal 117.

The first canal 115 is visible as an opening, a through hole, or an empty space according to the right side view of the device 100, as shown in FIG. IC. The second body 105 is not visible according to the right side view of the device 100. The first canal 115 is visible as an opening, a through hole, or an empty space according to the left side view of the device 100 (not shown in drawings). The first body 103 is not visible according to the left side view of the device 100.

In various embodiments, the device 100 is generally made of biocompatible materials, medical grade materials suitable for medical devices, or materials that are currently accepted by the U.S. Food and Drug Administration (FDA) for use in medical devices. For example, such materials include, but are not limited to silicones, polymers, metals, ceramics, rubber, plastics, or any combination thereof.

In various embodiments, the device 100 is configured to be coupled to a needle or an endo file to stop the needle or endo file during placement of the needle or endo file into a target site of a subject. The device 100 is configured to receive the needle or endo file such that the received needle or endo file may pass through the first canal. The first canal 115 has a ribbed surface 121 configured to grip the received need or endo file.

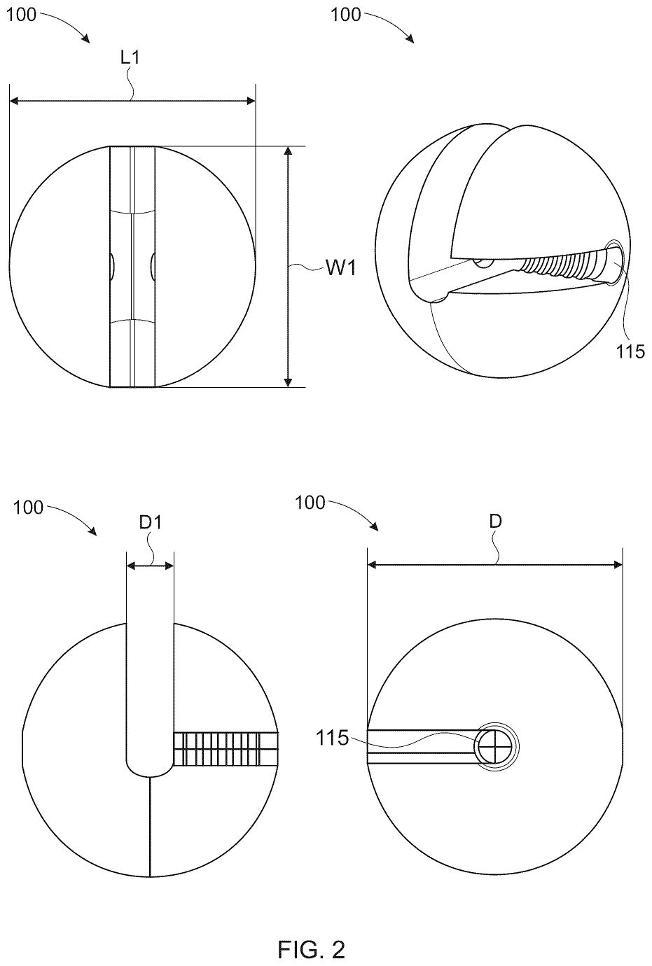

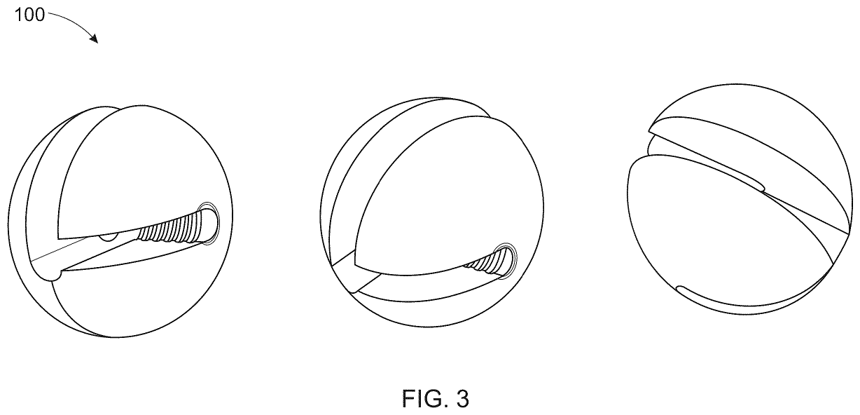

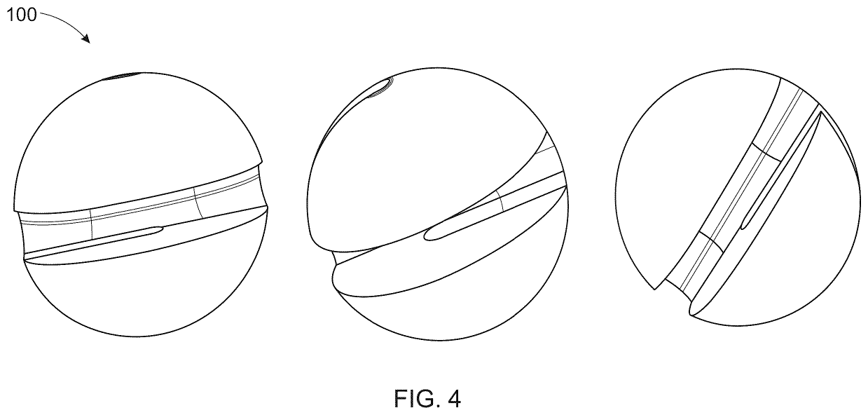

For example, FIG. 2 depicts a needle stop device 100. For example, the needle stop device 100 is for an RF needle. For example, the RF needle has a diameter of 1.5 millimeter (mm). For example, a diameter of an introductory or second canal 117 is 2.18 mm. For example, a diameter of a gripping or first canal 115 configured to receive the RF needle is at least 1.5 mm. However, the diameter of the gripping or first canal 115 is not limited to 1.5 mm. Based on the diameter of the RF needle to be used with the needle stop device 100, the diameter of the gripping or first canal 115 of the needle stop device 100 may be less or greater than 1.5 mm. For example, the gripping or first canal 115 has a ribbed surface like a ribbed condom. Although specific numbers are provided for various measurements, referring to FIG. 2, the numbers are merely exemplary and are not limited thereto. The numbers may be somewhat less or greater than the specific numbers, as provided herein.

In various embodiments, a diameter of the first canal 115 is at least about 0.5 millimeter (mm) and less than about 2.5 mm, about 0.5 mm, about 0.6 mm, about 0.7 mm, about 0.8 mm, about 0.9 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.1 mm, or about 2.5 mm.

In various embodiments, a diameter of the second canal 117 is at least about 1 millimeter (mm) and less than about 3 mm or 4 mm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.1 mm, about 2.5 mm, about 2.6 mm, about 2.7 mm, about 2.8 mm, about 2.9 mm, or about 3.0 mm.

The device 100 is further configured to stop the needle or endo file being inserted into the target site by blocking further insertion. For example, the needle includes a radiofrequency (RF) needle. For example, the endo file is a drill used in root canal surgery. The RF needle stop may be versatile in that it can either be coupled to the hub of the RF needle prior to skin puncture, or added independently after RF needle has been deployed. In various embodiments, the needle stop device is a generally round device. In various embodiments, a diameter (D) of the first body 103 and a diameter (D) of the second body 105 are same.

Referring to FIG. 2, in various embodiments, a distance (D1) between the first body 103 and the second body 105 generally corresponding to a width of the gap is at least about 1 millimeter (mm) and less than about 3 mm or 4 mm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.1 mm, about 2.5 mm, about 2.6 mm, about 2.7 mm, about 2.8 mm, about 2.9 mm, or about 3.0 mm. For example, the distance (D1) is 2.18 mm.

In various embodiments, a width (W1) of the device 100 generally corresponding to a length of the gap 107 is at least about 10 mm and less than about 13 mm, about 10 mm, about 10.2 mm, about 10.4 mm, about 10.6 mm, about 10.8 mm, about 11.0 mm, about 11.2 mm, about 11.4 mm, about 11.6 mm, about 11.8 mm, about 12.0 mm, about 12.2 mm, about 12.4 mm, about 12.6 mm, about 12.8 mm, or about 13.0 mm. For example, the width (W1) is 11.58 mm.

In various embodiments, a length (L1) of the device 100 is at least about 10 mm and less than about 13 mm, about 10 mm, about 10.2 mm, about 10.4 mm, about 10.6 mm, about 10.8 mm, about 11.0 mm, about 11.2 mm, about 11.4 mm, about 11.6 mm, about 11.8 mm, about 12.0 mm, about 12.2 mm, about 12.4 mm, about 12.6 mm, about 12.8 mm, or about 13.0 mm. For example, the length (L1) is 11.80 mm.

In various embodiments, the width (W1) and the length (L1) of the device 100 are different. For example, the width (W1) may be 11.58 mm and the length (L1) may be 11.80 mm. In certain embodiments, the width (W1) and the length (L1) of the device 100 are same. For example, both the width (W1) and the length (L1) may be 11.58 mm or 11.80 mm.

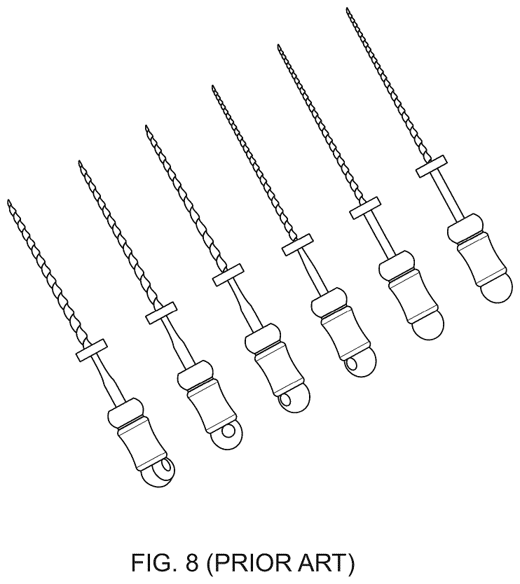

FIG. 8 shows a prior art device attached to endo file drills used in root canal surgery to be used as a stopper. Suitable examples of needle stops that may be utilized with the presently disclosed devices, systems and methods described herein will be apparent to a person of skill in the art.

### Anchor Sleeve

Provided are various embodiments of a device 200 used as an anchor sleeve for gripping along the entire length of the device, as compared to only a small section of the prior art anchor sleeves. The longer length of grip may translate into a much larger surface area of contact, which could improve pull out strength. The device 200 may be easily customizable with the anchors being easily removed or added to the wire/catheter at any point along the wire/catheter. In addition, multiple devices/anchor sleeves 200 may be added in series for additional security in placement. This may be of critical importance when implanting a patient with scoliosis where there is a larger risk for wire migration. Additional purchase may be critical in the cancer population for intrathecal catheter implantation due to poor tissue healing and reduced collagen production.

Titanium coating of the anchor lead may facilitate anchoring to a bony structure, improving stability further. The bony anchoring may be further enhanced with an expandable screw. One side of the anchor may have a flat or curved surface which may match better with a bony target for purchase.

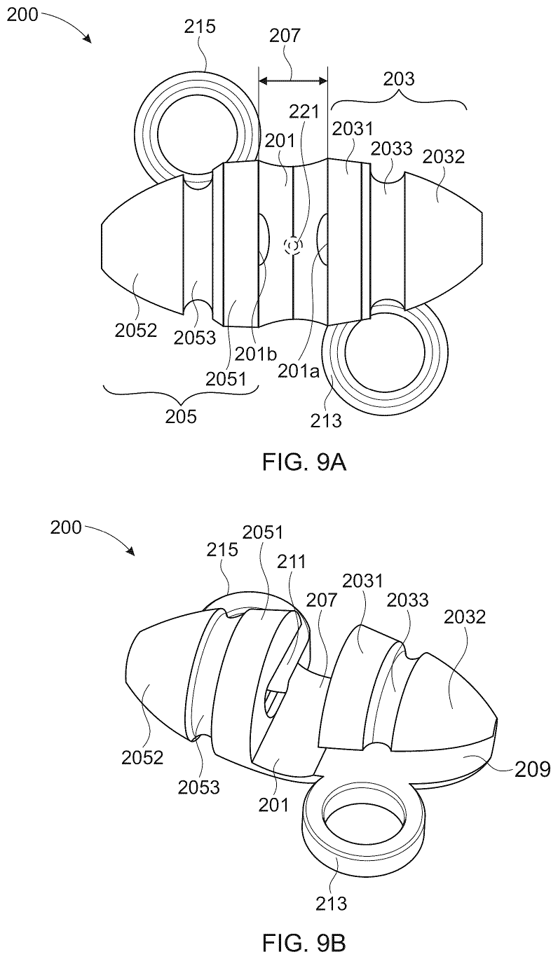

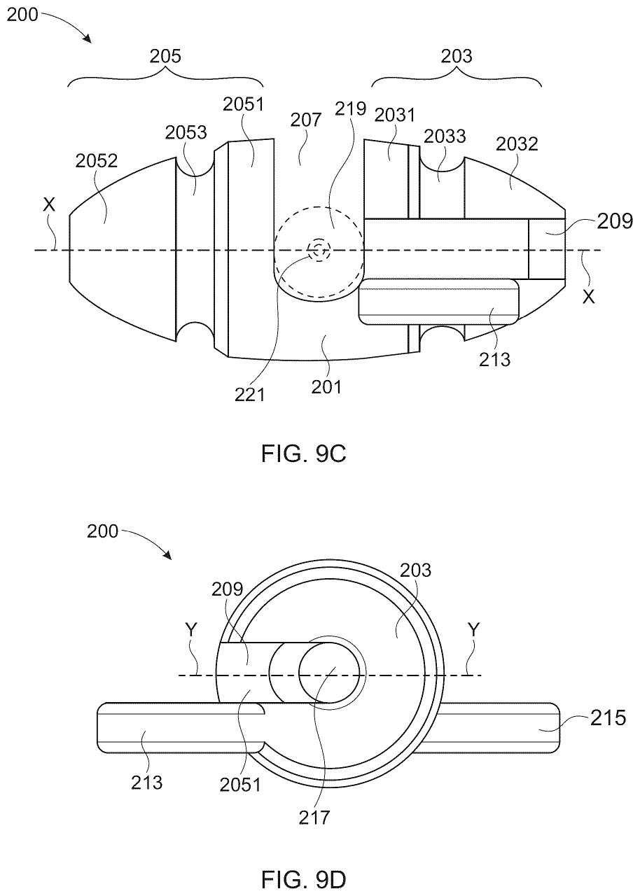

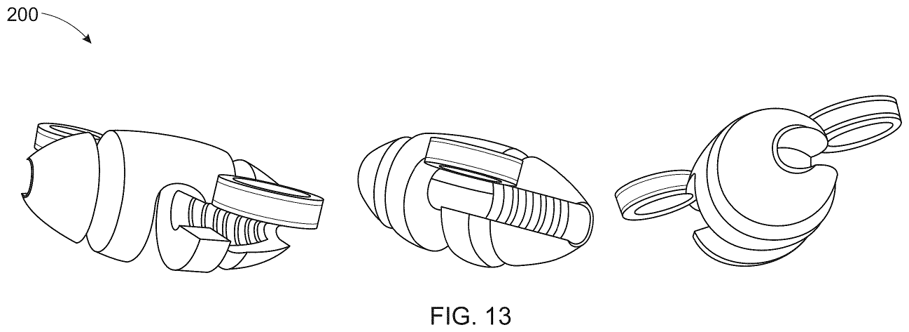

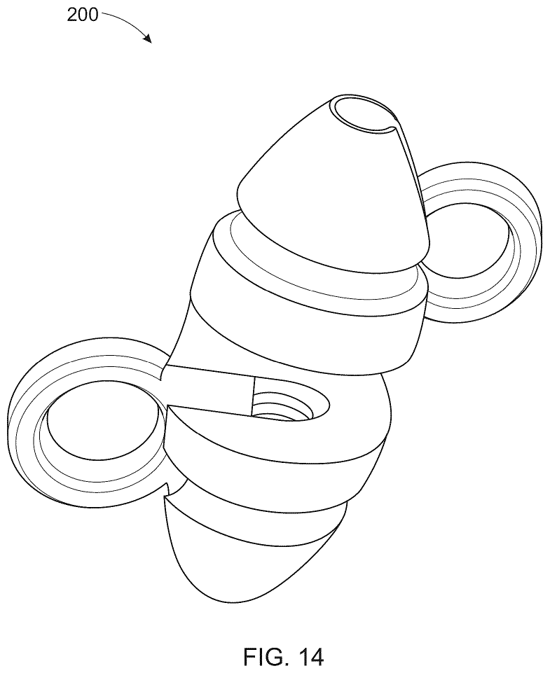

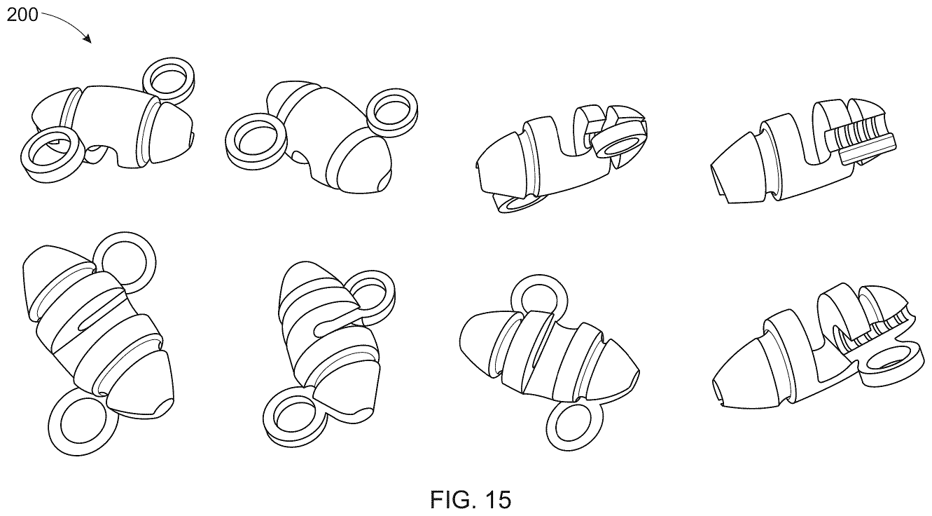

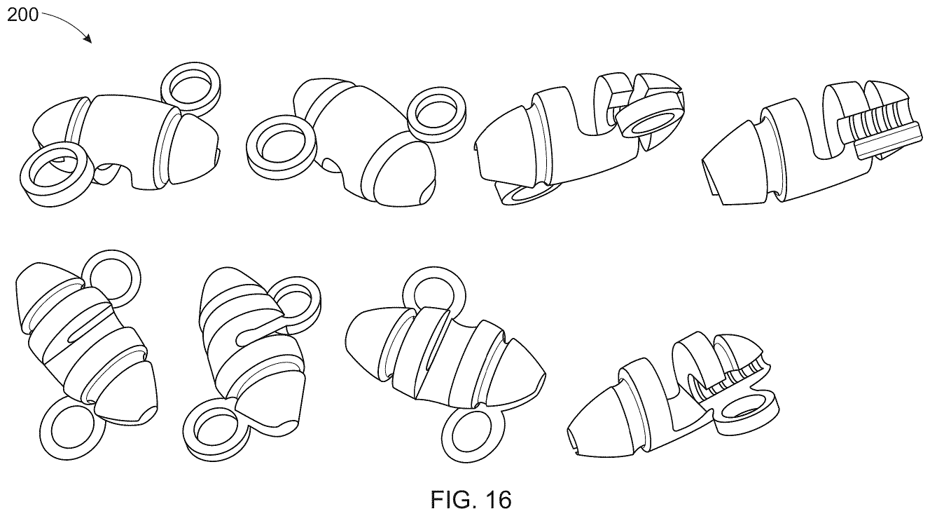

Referring to FIGS. 9A-16, in various embodiments, the device 200 includes a bridge 201, a first body 203, and a second body 205. The first body 203 may have a first body first portion 2031 connected to a first side 201a of the bridge 201. In some embodiments, the first body 203 further has a first body second portion 2032 connected to the first body first portion 2031 by a first body grooved portion 2033. The second body 205 has a second body first portion 2051 connected to a second side 201b of the bridge 201. In some embodiments, the second body 205 further has a second body second portion 2052 connected to the second body first portion 2051 by a second body grooved portion 2053.

The device 200 has a generally oval shape, or a general shape of the device 200 is similar to an oval, although other shapes and configurations may be used in alternate embodiments of the invention. The device 200 is elongated along a first axis X. A gap 207 is formed between the first body first portion 2031 and the second body first portion 2051.

The first body 203 further has a first slit 209 with an open space facing a first direction that is generally perpendicular to the first axis X. The second body 205 further has a second slit 211 with an open space facing a second direction that is generally perpendicular to the first axis X. The first direction is opposite the second direction.

In some embodiments, the device 200 further includes a first ring 213 coupled to the first body 203 and a second ring 215 coupled to the second body 205. The first ring 213 and the second ring 215 are used as suture holes. The first ring 213 is coupled to a side of the first body 203 at which the first slit 209 is formed, and the second ring 215 is coupled to a side of the second body 205 at which the second slit 211 is formed. The first ring 213 and the second ring 215 are positioned at opposite sides of the device 200 respect to the first axis X. A size of the first ring 213 and a size of the second ring 215 are same.

The device 200 is visible as a generally oval shape with two rings 213, 215 attached thereto or as a shape similar to an oval with two rings 213, 215 attached thereto according to a top view or a bottom view of the device 200, for example, as sown in FIG. 9A, although other shapes and configurations may be used in alternate embodiments of the invention. The first body 203, the bridge 201/gap 207, and the second body 205 are visible according to the top view of the device 200, as shown in FIG. 9A. The gap 207 is not visible according to the bottom view of the device 200 (not clearly shown in drawings). The bottom view is opposite the top view of the device 200 shown in FIG. 9A. The first slit 209 and the second slit 211 are not visible according to the top view or the bottom view of the device 200. The gap 207, the first ring 213, and the first slit 209 are visible, and the second slit 211 and the second ring 215 are not visible according to a front view of the device 200, as shown in FIG. 9C. The first body 203 and the second body 205 are visible according to the front view. The gap 207, the second ring 215, and the second slit 211 are visible, and the first slit 209 and the first ring 213 are not visible according to a rear view of the device 200 that is opposite the front view of the device 200 shown in FIG. 9C. The first body 203 and the second body 205 are visible according to the rear view. The first slit 209, the first ring 213, and the second ring 215 are visible according to a right side view of the device 200, as shown in FIG. 9D. The first body 203 are visible according to the right side view. While a portion of the second body first portion 2015 of the second body 205 are visible though the first slit 209, most of the second body 205 may not be visible according to the right side view. The device 200 is visible as a generally circular shape with two protrusions or as a shape similar to a circle with two protrusions according to the right side view of the device 200. The second slit 211, the first ring 213, and the second ring 215 are visible according to a left side view of the device 200 (not clearly shown in drawings) that is opposite the right side view of the device 200 shown in FIG. 9D. The second body 205 is visible and the first body 203 is not visible according to the left side view. The device 200 is visible as a generally circular shape with two protrusions or as a shape similar to a circle with two protrusions according to the left side view of the device 200.

The first slit 209 has an opening formed toward a first direction, and the second slit 211 has an opening formed toward a second direction that is opposite the first direction. A first open space is formed by a first portion of the gap 207 and the first slit 209, and a second open space is formed by a second portion of the gap 207 and the second slit 211. The first open space and the second open space overlap partially such that a first canal 217 is formed. The first canal 217 includes the overlapping space and passes centrally through the device 200 along the first axis X.

A top side of the bridge 201 is generally curved inwardly, as exemplified in FIG. 9C. A second canal 219 is formed on top of the bridge 201 and at a bottom portion of the gap 207. The second canal 219 is a portion of the gap 207 and passes centrally through the device 200 along a second axis Y (shown in FIG. 9D) that is generally perpendicular to the first axis X. The second canal 219 also includes the overlapping space and the first canal 217 is generally perpendicular to the second canal 219. The first canal 217 and the second canal 219 intersect each other at a point generally corresponding to a center 221 of the device 200.

The first slit 209 and the second slit 211 are positioned at opposite sides relative to the first canal 217. The first slit 209 and the second slit 211 are positioned at opposite sides relative to the second canal 219.

The first canal 217 is visible as an opening, a through hole, or an empty space according to the right side view of the device 200, as shown in FIG. 9D. The second body 205 is not visible according to the right side view of the device 200. The first canal 217 is visible as an opening, a through hole, or an empty space according to the left side view of the device 200. The first body 203 is not visible according to the left side view of the device 200. The first canal 217 is configured to be used as a gripping canal, and the second canal 219 is used as an introductory canal.

In various embodiments, the device 200 is generally made of biocompatible materials, medical grade materials suitable for medical devices, or materials that are currently accepted by the U.S. Food and Drug Administration (FDA) for use in medical devices. For example, such materials include, but are not limited to silicones, polymers, metals, ceramics, rubber, plastics, or any combination thereof.

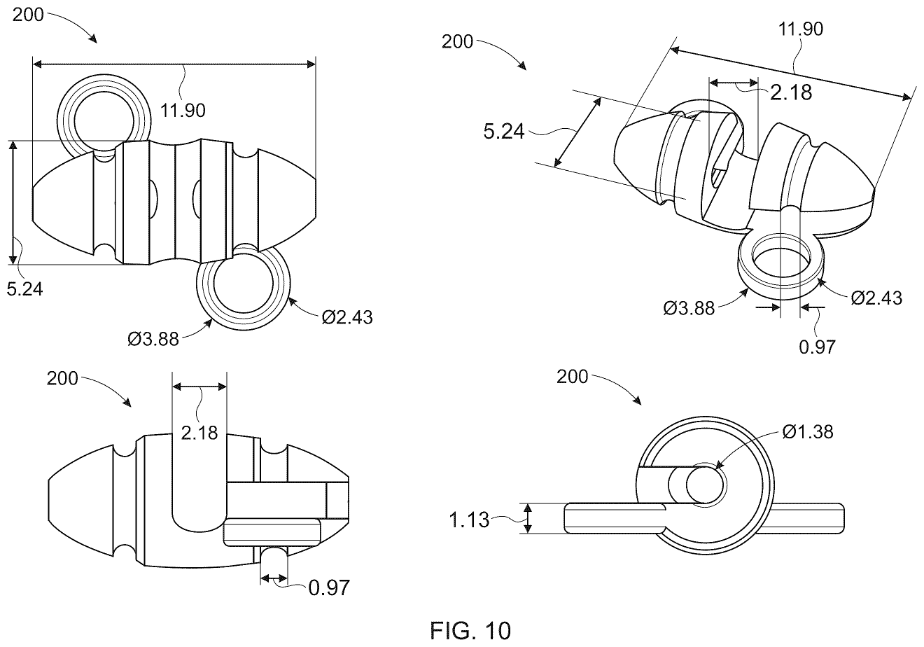

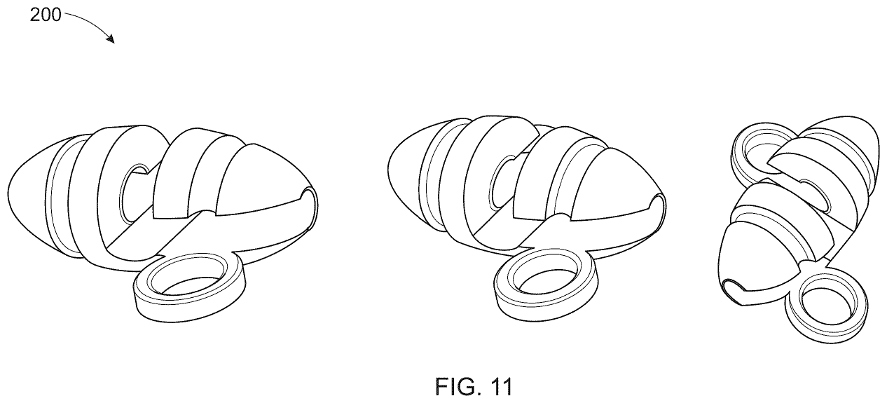

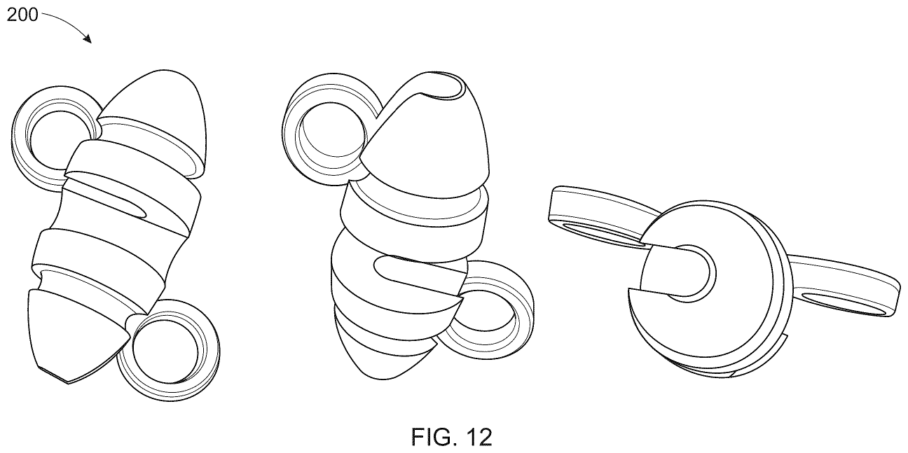

For example, referring to FIG. 10, the anchor sleeve device 200 is for a catheter of 1.38 mm diameter although the diameter of the catheter is not limited thereto. For example, the diameter of the introductory (second) canal 219 diameter is 2.18 mm. For example, the diameter of the gripping (first) canal 217 is 1.38 mm. The gripping (first) canal 217 has a ribbed surface like a ribbed condom. For example, grooves for suture are 0.97 mm diameter.

In various embodiments, an inner diameter of the first ring 213 or second ring 215 is at least about 2.0 millimeter (mm) and less than about 3.0 mm, about 2.0 mm, about 2.2 mm, about 2.4 mm, about 2.6 mm, about 2.8 mm, or about 3.0 mm. For example, the inner diameter of the first ring 213 or second ring 215 is 2.43 mm, as shown in FIG. 10.

In various embodiments, an outer diameter of the first ring 213 or second ring 215 is at least about 3.0 millimeter (mm) and less than about 4.6 mm, about 3.0 mm, about 3.2 mm, about 3.4 mm, about 3.6 mm, about 3.8 mm, about 4.0 mm, about 4.2 mm, about 4.4 mm, or about 4.6 mm. For example, the outer diameter of the first ring 213 or second ring 215 is 3.88 mm, as shown in FIG. 10.

In various embodiments, a thickness of the first ring 213 or second ring 215 is at least about 0.8 millimeter (mm) and less than about 2.0 mm, about 0.8 mm, about 0.9 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, or about 2.0 mm. For example, the thickness of the first ring 213 or second ring 215 is 1.13 mm, as shown in FIG. 10.

In various embodiments, a distance between the first body 203 and the second body 205 generally corresponding to a width of the gap 207 is least about 1.8 millimeter (mm) and less than about 3 mm, about 1.8 mm, about 1.9 mm, about 2.0 mm, about 2.1 mm, about 2.2 mm, about 2.3 mm, about 2.4 mm, about 2.5 mm, about 2.6 mm, about 2.7 mm, about 2.8 mm, about 2.9 mm, or about 3.0 mm. For example, the distance between the first body 203 and the second body 205 is 2.18 mm, as shown in FIG. 10.

In various embodiments, a length of the gap 207 is least about 4.5 millimeter (mm) and less than about 7.0 mm, about 4.5 mm, about 5.0 mm, about 5.5 mm, about 6.0 mm, about 6.5 mm, or about 7.0 mm. For example, the length of the gap 207 is 5.24 mm, as shown in FIG. 10.

In various embodiments, a length of the device 200 is about at least 10.0 millimeter (mm) and less than about 13 mm, about 10.0 mm, about 10.2 mm, about 10.4 mm, about 10.6 mm, about 10.8 mm, about 11.0 mm, about 11.2 mm, about 11.4 mm, about 11.6 mm, about 11.8 mm, about 12.0 mm, about 12.2 mm, about 12.4 mm, about 12.6 mm, about 12.8 mm, or about 13.0 mm. For example, the length of the device 200 is 11.90 mm, as shown in FIG. 10.

In various embodiments, a diameter of the first canal 217 is at least about 0.8 millimeter (mm) and less than about 2.0 mm, about 0.8 mm, about 0.9 mm, about 1.0 mm, about 1.2 mm, about 1.3 mm, about 1.4 mm, about 1.5 mm, about 1.6 mm, about 1.7 mm, about 1.8 mm, about 1.9 mm, or about 2.0 mm. For example, the diameter of the first canal 217 is 1.38 mm, as shown in FIG. 10. Although specific numbers are provided for various measurements, referring to FIG. 10, the numbers are merely exemplary and are not limited thereto. The numbers may be somewhat less or greater than the specific numbers, as provided hercin.

In various embodiments, a first groove 2033 is formed radially between the first body first portion 2031 and the first body second portion 2032. A second groove 2053 is formed radially between the second body first portion 2051 and the second body second portion 2052.

In various embodiments, a first distance between the first body first portion 2031 and the first body second portion 2032 and a second distance between the second body first portion 2051 and the second body second portion 2052 are same. In various embodiments, the first or second distance generally corresponding to a width of the first groove 2033 or second groove 2053 is at least about 0.5 millimeter (mm) and less than about 1.5 mm, about 0.5 mm, about 0.6 mm, about 0.7 mm, about 0.8 mm, about 0.9 mm, about 1.0 mm, about 1.2 mm, about 1.3 mm, about 1.4 mm, or about 1.5 mm. For example, each of the first distance between the first body first portion 2031 and the first body second portion 2032 and the second distance between the second body first portion 2051 and the second body second portion 2052 is 0.97 mm, as shown in FIG. 10.

The device 200 is for use as a single piece anchor sleeve. The device 200 is configured to grab a lead by gripping along the entire length of the device 200. The device 200 is further configured to snap into place, minimizing lead damage and improving pullout strength. The device 200 is configured to secure lead placement for a pacemaker, an implantable cardioverter-defibrillator (ICD), a spinal cord stimulator lead, or intrathecal drug delivery catheter, preventing lead dislodgement.

### EXEMPLARY EMBODIMENTS

Various embodiments of the present invention provide a device including a bridge; a first body connected to a first side of the bridge, the first body having a generally hemisphere shape or a shape similar to a hemisphere such that the first body includes a first surface that is curved and a second surface that is flat and having a shape of a circle; and a second body connected to a second side of the bridge, the second body having a generally hemisphere shape or a shape similar to a hemisphere such that the second body includes a third surface that is curved and a fourth surface that is flat and having a shape of a circle. A gap is formed on top of the bridge and in-between the second surface of the first body and the fourth surface of the second body.

In some embodiments, the overall shape of the device is generally spherical, or a general shape of the device is similar to a sphere.

In some embodiments, the first body has a first slit and the second body has a

second slit.

In some embodiments, the device is visible as a circle or as a shape similar to a circle according to a top view or a bottom view of the device.

In some embodiments, the first body, the gap, and the second body are visible according to the top view of the device.

In some embodiments, the gap is not visible according to the bottom view of the device, the bottom view being opposite the top view of the device.

In some embodiments, the first slit and the second slit are not visible according to the top view or the bottom view of the device.

In some embodiments, the gap and the first slit are visible and the second slit is not visible according to a front view of the device, the first body and the second body being visible according to the front view.

In some embodiments, the gap and the second slit are visible and the first slit is not visible according to a rear view of the device that is opposite the front view of the device, the first body and the second body being visible according to the rear view.

In some embodiments, the entire first slit is visible and the second slit is not visible entirely according to a right side view of the device, the first body being visible and the second body not being visible according to the right side view.

In some embodiments, the device is visible as a circle or as a shape similar to a circle according to the right side view of the device.

In some embodiments, the entire second slit is visible and the first slit is not visible entirely according to a left side view of the device that is opposite the right side view of the device, the second body being visible and the first body not being visible according to the left side view.

In some embodiments, the device is visible as a circle or as a shape similar to a circle according to the left side view of the device.

In some embodiments, the first slit has an opening formed toward a first direction and the second slit has an opening formed toward a second direction that is opposite the first direction.

In some embodiments, a first open space is formed by a first portion of the gap and the first slit and a second open space is formed by a second portion of the gap and the second slit.

In some embodiments, the first open space and the second open space overlap partially such that a first canal is formed, the first canal including the overlapping space and passing centrally through the device.

In some embodiments, a top side of the bridge is curved inwardly such that a second canal is formed on top of the bridge and at a bottom portion of the gap, the second canal being a portion of the gap and passing centrally through the device.

In some embodiments, the second canal also includes the overlapping space and the first canal is perpendicular to the second canal.

In some embodiments, the first canal and the second canal intersect each other at a point corresponding to a center of the device, the device generally having a shape of a sphere or an overall shape of the device being similar to a sphere.

In some embodiments, the first slit and the second slit are positioned at opposite sides relative to the first canal.

In some embodiments, the first slit and the second slit are positioned at opposite sides relative to the second canal.

In some embodiments, the first canal is visible as an opening, a through hole, or an empty space according to the right side view of the device, the second body not being visible according to the right side view of the device.

In some embodiments, the first canal is visible as an opening, a through hole, or an empty space according to the left side view of the device, the first body not being visible according to the left side view of the device.

In some embodiments, the device is made of biocompatible materials, medical grade materials suitable for medical devices, or materials that are currently accepted by the U.S. Food and Drug Administration (FDA) for use in medical devices, the materials including, but not limited to silicones, polymers, metals, ceramics, rubber, plastics, or any combination thereof.

In some embodiments, the device is configured to be coupled to a needle or an endo file to stop the needle or endo file during placement of the needle or endo file into a target site of a subject.

In some embodiments, the device is configured to receive the needle or endo file, the received needle or endo file passing through the first canal (of claim 16).

In some embodiments, the first canal has a ribbed surface configured to grip the received need or endo file.

In some embodiments, a diameter of the first canal is at least about 0.5 millimeter (mm) and less than about 2.5 mm, about 0.5 mm, about 0.6 mm, about 0.7 mm, about 0.8 mm, about 0.9 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.1 mm, or about 2.5 mm.

In some embodiments, a diameter of the second canal is at least about 1 millimeter (mm) and less than about 3 mm or 4 mm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.1 mm, about 2.5 mm, about 2.6 mm, about 2.7 mm, about 2.8 mm, about 2.9 mm, or about 3.0 mm.

In some embodiments, the device is further configured to stop the needle or endo file being inserted into the target site by blocking further insertion.

In some embodiments, the needle comprises a radiofrequency (RF) needle.

In some embodiments, the endo file is a drill used in root canal surgery.

In some embodiments, a diameter of the first body and a diameter of the second body are same.

In some embodiments, a distance between the first side body and the second side body corresponding to a width of the gap is at least about 1 millimeter (mm) and less than about 3 mm or 4 mm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.1 mm, about 2.5 mm, about 2.6 mm, about 2.7 mm, about 2.8 mm, about 2.9 mm, or about 3.0 mm.

In some embodiments, a width of the device corresponding to a length of the gap is at least about 10 mm and less than about 13 mm, about 10 mm, about 10.2 mm, about 10.4 mm, about 10.6 mm, about 10.8 mm, about 11.0 mm, about 11.2 mm, about 11.4 mm, about 11.6 mm, about 11.8 mm, about 12.0 mm, about 12.2 mm, about 12.4 mm, about 12.6 mm, about 12.8 mm, or about 13.0 mm.

In some embodiments, a length of the device is at least about 10 mm and less than about 13 mm, about 10 mm, about 10.2 mm, about 10.4 mm, about 10.6 mm, about 10.8 mm, about 11.0 mm, about 11.2 mm, about 11.4 mm, about 11.6 mm, about 11.8 mm, about 12.0 mm, about 12.2 mm, about 12.4 mm, about 12.6 mm, about 12.8 mm, or about 13.0 mm.

In some embodiments, the width and the length of the device are different.

In some embodiments, the width and the length of the device are same.

Additional embodiments include those listed below:

Embodiment 1. A device including:
a bridge;
a first body having a first body first portion connected to a first side of the bridge, the first body further having a first body second portion connected to the first body first portion by a first body grooved portion; and
a second body having a second body first portion connected to a second side of the bridge, the second body further having a second body second portion connected to the second body first portion by a second body grooved portion.

Embodiment 2. The device of Embodiment 1, wherein the device generally has an oval shape, or a general shape of the device is similar to an oval, the device elongated along a first axis.

Embodiment 3. The device of Embodiment 1, wherein a gap is formed between the first body first portion and the second body first portion.

Embodiment 4. The device of Embodiment 1, wherein the first body further has a first slit with an open space facing a first direction that is perpendicular to the first axis.

Embodiment 5. The device of Embodiment 4, wherein the second body further has a second slit with an open space facing a second direction that is perpendicular to the first axis, the first direction being opposite the second direction.

Embodiment 6. The device of Embodiment 5, further comprising a first ring coupled to the first body and a second ring coupled to the second body.

Embodiment 7. The device of Embodiment 6, wherein the first ring and the second ring are used as suture holes.

Embodiment 8. The device of Embodiment 6, wherein the first ring is coupled to a side of the first body at which the first slit is formed, and the second ring is coupled to a side of the second body at which the second slit is formed.

Embodiment 9. The device of Embodiment 6, wherein the first ring and the second ring are positioned at opposite sides of the device respect to the first axis.

Embodiment 10. The device of claim 6, wherein a size of the first ring and a size of the second ring are same.

Embodiment 11. The device of any one of Embodiments 1-10, wherein the device is visible as a generally oval shape with two rings attached thereto or as a shape similar to an oval with two rings attached thereto according to a top view or a bottom view of the device.

Embodiment 12. The device of any one of Embodiments 1-11, wherein the first body, the gap, and the second body are visible according to the top view of the device.

Embodiment 13. The device of any one of Embodiments 1-12, wherein the gap is not visible according to the bottom view of the device, the bottom view being opposite the top view of the device.

Embodiment 14. The device of any one of Embodiments 1-13, wherein the first slit and the second slit are not visible according to the top view or the bottom view of the device.

Embodiment 15. The device of any one of Embodiments 1-14, wherein the gap, the first ring, and the first slit are visible, and the second slit and the second ring are not visible according to a front view of the device, the first body and the second body being visible according to the front view.

Embodiment 16. The device of any one of Embodiments 1-15, wherein the gap, the second ring, and the second slit are visible, and the first slit and the first ring are not visible according to a rear view of the device that is opposite the front view of the device, the first body and the second body being visible according to the rear view.

Embodiment 17. The device of any one of Embodiments 1-16, wherein the first slit, the first ring, and the second ring are visible according to a right side view of the device, the first body being visible and most of the second body not being visible according to the right side view while a portion of the second body is visible through the first slit according to the right side view.

Embodiment 18. The device of any one of Embodiments 1-17, wherein the device is visible as a generally circular shape with two protrusions or as a shape similar to a circle with two protrusions according to the right side view of the device.

Embodiment 19. The device of any one of Embodiments 1-18, wherein the second slit, the first ring, and the second ring are visible according to a left side view of the device that is opposite the right side view of the device, the second body being visible and the first body not being visible according to the left side view.

Embodiment 20. The device of any one of Embodiments 1-19, wherein the device is visible as a generally circular shape with two protrusions or as a shape similar to a circle with two protrusions according to the left side view of the device.

Embodiment 21. The device of any one of Embodiments 1-20, wherein the first slit has an opening formed toward a first direction and the second slit has an opening formed toward a second direction that is opposite the first direction.

Embodiment 22. The device of any one of Embodiments 1-21, wherein a first open space is formed by a first portion of the gap and the first slit and a second open space is formed by a second portion of the gap and the second slit.

Embodiment 23. The device of any one of Embodiments 1-22, wherein the first open space and the second open space overlap partially such that a first canal is formed, the first canal including the overlapping space and passing centrally through the device along the first axis.

Embodiment 24. The device of any one of Embodiments 1-23, wherein a top side of the bridge is curved inwardly such that a second canal is formed on top of the bridge and at a bottom portion of the gap, the second canal being a portion of the gap and passing centrally through the device along a second axis that is perpendicular to the first axis.

Embodiment 25. The device of any one of Embodiments 1-24, wherein the second canal also includes the overlapping space and the first canal is perpendicular to the second canal.

Embodiment 26. The device of any one of Embodiments 1-25, wherein the first canal and the second canal intersect each other at a point corresponding to a center of the device.

Embodiment 27. The device of any one of Embodiments 1-26, wherein the first slit and the second slit are positioned at opposite sides relative to the first canal.

Embodiment 28. The device of any one of Embodiments 1-27, wherein the first slit and the second slit are positioned at opposite sides relative to the second canal.

Embodiment 29. The device of any one of Embodiments 1-28, wherein the first canal is visible as an opening, a through hole, or an empty space according to the right side view of the device, the second body not being visible according to the right side view of the device.

Embodiment 30. The device of any one of Embodiments 1-29, wherein the first canal is visible as an opening, a through hole, or an empty space according to the left side view of the device, the first body not being visible according to the left side view of the device.

Embodiment 31. The device of any one of Embodiments 1-30, wherein the first canal is used as a gripping canal and the second canal is used as an introductory canal.

Embodiment 32. The device of any one of Embodiments 1-31, wherein the device is made of biocompatible materials, medical grade materials suitable for medical devices, or materials that are currently accepted by the U.S. Food and Drug Administration (FDA) for use in medical devices, the materials including, but not limited to silicones, polymers, metals, ceramics, rubber, plastics, or any combination thereof.

Embodiment 33. The device of any one of Embodiments 1-32, wherein an inner diameter of the first or second ring is at least about 2.0 millimeter (mm) and less than about 3.0 mm, about 2.0 mm, about 2.2 mm, about 2.4 mm, about 2.6 mm, about 2.8 mm, or about 3.0 mm.

Embodiment 34. The device of any one of Embodiments 1-33, wherein an outer diameter of the first or second ring is at least about 3.0 millimeter (mm) and less than about 4.6 mm, about 3.0 mm, about 3.2 mm, about 3.4 mm, about 3.6 mm, about 3.8 mm, about 4.0 mm, about 4.2 mm, about 4.4 mm, or about 4.6 mm.

Embodiment 35. The device of any one of Embodiments 1-34, wherein a thickness of the first or second ring is at least about 0.8 millimeter (mm) and less than about 2.0 mm, about 0.8 mm, about 0.9 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, or about 2.0 mm.

Embodiment 36. The device of any one of Embodiments 1-35, wherein a distance between the first body and the second body corresponding to a width of the gap is least about 1.8 millimeter (mm) and less than about 3 mm, about 1.8 mm, about 1.9 mm, about 2.0 mm, about 2.1 mm, about 2.2 mm, about 2.3 mm, about 2.4 mm, about 2.5 mm, about 2.6 mm, about 2.7 mm, about 2.8 mm, about 2.9 mm, or about 3.0 mm.

Embodiment 37. The device of any one of Embodiments 1-36, wherein a length of the gap is least about 4.5 millimeter (mm) and less than about 7.0 mm, about 4.5 mm, about 5.0 mm, about 5.5 mm, about 6.0 mm, about 6.5 mm, or about 7.0 mm.

Embodiment 38. The device of any one of Embodiments 1-37, wherein a length of the device is about at least 10.0 millimeter (mm) and less than about 13 mm, about 10.0 mm, about 10.2 mm, about 10.4 mm, about 10.6 mm, about 10.8 mm, about 11.0 mm, about 11.2 mm, about 11.4 mm, about 11.6 mm, about 11.8 mm, about 12.0 mm, about 12.2 mm, about 12.4 mm, about 12.6 mm, about 12.8 mm, or about 13.0 mm.

Embodiment 39. The device of any one of Embodiments 1-38, wherein a diameter of the first canal is at least about 0.8 millimeter (mm) and less than about 2.0 mm, about 0.8 mm, about 0.9 mm, about 1.0 mm, about 1.2 mm, about 1.3 mm, about 1.4 mm, about 1.5 mm, about 1.6 mm, about 1.7 mm, about 1.8 mm, about 1.9 mm, or about 2.0 mm.

Embodiment 40. The device of any one of Embodiments 1-39, wherein a first groove is formed radially between the first body first portion and the first body second portion, and a second groove is formed radially between the second body first portion and the second body second portion.

Embodiment 41. The device of any one of Embodiments 1-40, wherein a first distance between the first body first portion and the first body second portion and a second distance between the second body first portion and the second body second portion are same.

Embodiment 42. The device of any one of Embodiments 1-41, wherein the first or second distance corresponding to a width of the first or second groove is at least about 0.5 millimeter (mm) and less than about 1.5 mm, about 0.5 mm, about 0.6 mm, about 0.7 mm, about 0.8 mm, about 0.9 mm, about 1.0 mm, about 1.2 mm, about 1.3 mm, about 1.4 mm, or about 1.5 mm.

Embodiment 43. The device of any one of Embodiments 1-42, wherein the device is for use as a single piece anchor sleeve.

Embodiment 44. The device of any one of Embodiments 1-43, wherein the device is configured to grab a lead by gripping along the entire length of the device.

Embodiment 45. The device of any one of Embodiments 1-44, wherein the device is further configured to snap into place, minimizing lead injury and improving pullout strength.

Embodiment 46. The device of any one of Embodiments 1-45, wherein the device is configured to secure lead placement for a pacemaker, an implantable cardioverter-defibrillator (ICD), a spinal cord stimulator lead, or intrathecal drug delivery catheter, preventing lead dislodgement.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Preferred embodiments of this application are described herein, including the best mode known to the inventors for carrying out the application. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

It is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

1. A device comprising:
a bridge;
a first body connected to a first side of the bridge, the first body having a generally hemisphere shape and including a first surface and a second surface, the first surface being a non-flat curved surface, the second surface being in the form of a flat circle; and
a second body connected to a second side of the bridge, the second body having a generally hemisphere shape and including a third surface and a fourth surface, the third surface being a non-flat curved surface, the fourth surface being in the form of a flat circle;
wherein a gap is formed on top of the bridge and in-between the second surface of the first body and the fourth surface of the second body, the gap being configured for receiving within at least a portion of a medical device; and
wherein, when the medical device is at least partially inserted into a target site of a subject, at least one of the first body and the second body prevents contact between (i) at least a portion of the medical device and (ii) the target site.

2. The device of claim 1, wherein the overall shape of the device is generally spherical, and wherein the device is visible as a circular shape according to a top view or a bottom view of the device.

3. The device of claim 1, wherein the first body, the gap, and the second body are visible according to a top view of the device, and wherein the gap is not visible according to a bottom view of the device, the bottom view being opposite the top view of the device.

4. The device of claim 3, wherein the first body has a first slit and the second body has a second slit.

5. The device of claim 4, wherein the first slit and the second slit are not visible according to the top view or the bottom view of the device.

6. The device of claim 5, wherein:
the gap and the first slit are visible and the second slit is not visible according to a front view of the device, the first body and the second body being visible according to the front view; and
the gap and the second slit are visible and the first slit is not visible according to a rear view of the device that is opposite the front view of the device, the first body and the second body being visible according to the rear view.

7. The device of claim 6, wherein:
the entire first slit is visible and the second slit is not visible entirely according to a right side view of the device, the first body being visible and the second body not being visible according to the right side view;
the device is visible as a circle or as a shape similar to a circle according to the right side view of the device;
the entire second slit is visible and the first slit is not visible entirely according to a left side view of the device that is opposite the right side view of the device, the second body being visible and the first body not being visible according to the left side view; and
the device is visible as a circle or as a shape similar to a circle according to the left side view of the device.

8. The device of claim 4, wherein:
the first slit has an opening formed toward a first direction and the second slit has an opening formed toward a second direction that is opposite the first direction; and
a first open space is formed by a first portion of the gap and the first slit and a second open space is formed by a second portion of the gap and the second slit.

9. The device of claim 8, wherein:
the first open space and the second open space overlap partially such that a first canal is formed, the first canal including the overlapping space and passing centrally through the device;
a top side of the bridge is curved inwardly such that a second canal is formed on top of the bridge and at a bottom portion of the gap, the second canal being a portion of the gap and passing centrally through the device;
the second canal also includes the overlapping space and the first canal is perpendicular to the second canal;
the first canal and the second canal intersect each other at a point corresponding to a center of the device, the device generally having a shape of a sphere or an overall shape of the device being similar to a sphere; and
the first slit and the second slit are positioned at opposite sides relative to the first canal.

10. The device of claim 9, wherein the first slit and the second slit are positioned at opposite sides relative to the second canal.

11. The device of claim 10, wherein:
the first canal is visible as an opening, a through hole, or an empty space according to the right side view of the device, the second body not being visible according to the right side view of the device; and
the first canal is visible as an opening, a through hole, or an empty space according to the left side view of the device, the first body not being visible according to the left side view of the device.

12. The device of claim 9, wherein:
the medical device includes a needle or an endo file;
the device is configured to receive the needle or endo file such that the received needle or endo file passes through the first canal; and
the first canal has a ribbed surface configured to grip the received need or endo file.

13. The device of claim 9, wherein a diameter of the first canal is at least about 0.5 millimeter (mm) and less than about 2.5 mm, about 0.5 mm, about 0.6 mm, about 0.7 mm, about 0.8 mm, about 0.9 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.1 mm, or about 2.5 mm.

14. The device of claim 13, wherein a diameter of the second canal is at least about 1 millimeter (mm) and less than about 3 mm or 4 mm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.1 mm, about 2.5 mm, about 2.6 mm, about 2.7 mm, about 2.8 mm, about 2.9 mm, or about 3.0 mm.

15. The device of claim 12, wherein the device is further configured to stop the needle or endo file being inserted into the target site by blocking further insertion.

16. The device of claim 12, wherein:
the needle comprises a radiofrequency (RF) needle; or
the endo file is a drill used in root canal surgery.

17. The device of claim 1, wherein:
a diameter of the first body and a diameter of the second body are same; and/or
a width of the device and a length of the device are same.

18. The device of claim 1, wherein a distance between a first side body and a second side body corresponding to a width of the gap is at least about 1 millimeter (mm) and less than about 3 mm or 4 mm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.1 mm, about 2.5 mm, about 2.6 mm, about 2.7 mm, about 2.8 mm, about 2.9 mm, or about 3.0 mm.

19. The device of claim 1, wherein:
a width of the device corresponding to a length of the gap is at least about 10 mm and less than about 13 mm, about 10 mm, about 10.2 mm, about 10.4 mm, about 10.6 mm, about 10.8 mm, about 11.0 mm, about 11.2 mm, about 11.4 mm, about 11.6 mm, about 11.8 mm, about 12.0 mm, about 12.2 mm, about 12.4 mm, about 12.6 mm, about 12.8 mm, or about 13.0 mm; and
a length of the device is at least about 10 mm and less than about 13 mm, about 10 mm, about 10.2 mm, about 10.4 mm, about 10.6 mm, about 10.8 mm, about 11.0 mm, about 11.2 mm, about 11.4 mm, about 11.6 mm, about 11.8 mm, about 12.0 mm, about 12.2 mm, about 12.4 mm, about 12.6 mm, about 12.8 mm, or about 13.0 mm.

20. The device of claim 1, wherein the device is made of biocompatible materials, medical grade materials suitable for medical devices, or materials that are currently accepted by the U.S. Food and Drug Administration (FDA) for use in medical devices, the materials including, but not limited to silicones, polymers, metals, ceramics, rubber, plastics, or any combination thereof.
